(19) United States
(12) Patent Application Publication
(10) Pub. No.: US 2008/0009698 A1
(43) Pub. Date: Jan. 10, 2008

(54) Method and device for visualizing objects

(75) Inventors: Jan Boese, Eckental (DE); Estelle Camus, Mountain View, CA (US); Marcus Pfister, Bubenreuth (DE); Thomas Redel, Poxdorf (DE)

Correspondence Address: SIEMENS CORPORATION;INTELLECTUAL PROPERTY DEPARTMENT, 170 WOOD AVENUE SOUTH, ISELIN, NJ, 08830, US

(73) Assignee: SIEMENS AKTIENGESELLSCHAFT

(21) Appl. No.: 11/805,216

(22) Filed: May 22, 2007

(30) Foreign Application Priority Data
May 22, 2006 (DE) 10 2006 024 000.6

(51) Int. Cl. A61B 5/05

(52) U.S. Cl. 600/407

(57) ABSTRACT

The present invention relates to a method and to a device for visualizing objects, in particular non-rigid objects. The method and the device are particularly suitable to visualizing three-dimensional objects in the case of medical interventions.

The method comprises:
providing a three-dimensional image data record of the object,
successively taking a series of two-dimensional image data records of the object,
individually registering each individual two-dimensional image data record with the three-dimensional image data record,
functionally evaluating functional parameters from the successively taken two-dimensional images,
extracting two-dimensional projections from the three-dimensional image data record, and
superimposing the recorded two-dimensional images with the extracted two-dimensional projections.

A clean copy of the abstract that incorporates the above amendments is provided herewith on a separate page.

### CROSS REFERENCE TO RELATED APPLICATIONS

This application claims priority of German application No. 10 2006 024 000.6 filed May 22, 2006, which is incorporated by reference herein in its entirety.

### FIELD OF THE INVENTION

The present invention relates to a method and to a device for visualizing objects, in particular non-rigid objects. The method and the device are particularly suitable for visualizing three-dimensional objects in the case of medical interventions.

### BACKGROUND OF THE INVENTION

The angiographic depiction of coronary arteries and calculation of the diameter, number and length of these arteries is currently one of the most important diagnostic aids in cardiology. Additional functional information, such as myocardial perfusion or determination of the flow rate, are further items of information which, in principle, can be obtained by way of angiography.

A method for X-ray projection-based perfusion imaging is known. In principle a series of images is created, which images are successively taken. This method can in principle also be used for any perfused tissue in other organs. The change in gray scale values or the change over time in gray scale values is substantially analyzed in a parcel (image section) of the myocardium while administering contrast medium. For this purpose one or more images are taken at defined phases of the cardiac cycle over a plurality of cardiac cycles, the patient holding his breath where possible. FIG. 4 shows a view of a two-dimensional series of images of a heart, the individual images being slightly displaced relative to each other by respiratory movements or movements of the heart. This can lead to errors in the case of an automatic “TIMI blush evaluation”.

In general the object of these functional evaluations is to compare images from different cardiac cycles with each other and obtain the functional information from the progression of changes in gray scale values over time. One problem in this connection however is that even if the individual images of the different cardiac cycles are taken in phase (for example ECG-triggered or by retrospective ECG triggering), the heart and therewith the coronary arteries and the perfused myocardium are located at slightly different positions with spatially different orientations.

Specifically, this “movement” of the coronary arteries generates an eraser effect in the known method for X-ray projection-based perfusion imaging. Since in this case the pixels, which are described as the vascular object during the visibility of the coronary arteries, are excluded from further evaluation, a large region that cannot be evaluated is produced.

It is important that the contrast medium progression in the individual picture areas (pixels) can be followed as well as possible. For this purpose it is important to ensure that the respective corresponding regions in the various images also actually lie one on top of the other, irrespective of movements of the object, for instance with slight respiratory movements or movements of the heart.

3D imaging of the heart is possible nowadays with the aid of computed tomography/DynaCT/MRI. In particular, depiction of the coronary arteries is perfectly feasible using CT angiography. However, owing to the different coronary artery branches, it is not possible to reach conclusions on perfusion of the myocardium using CT/DynaCT. A combined evaluation of the morphological 3D data (CT/DynaCT) and the interventionally obtained two-dimensional, functional information from angiography in the catheter laboratory would be ideal.

From Malsh, Dickhaus and Kücherer, “Quantitative Analyse von koronarangiographischen Bildfolgen zur Bestimmung der Myokardperfusion” Proceedings of the Workshop Bildverarbeitung in der Medizin 2003, Erlangen an approach is known which uses digital subtraction images as the basis for an evaluation. Here a procedure is described which compensates slight movement artifacts by way of diaphragm movement and compares manually defined anchoring points on the diaphragm between images. The mask image is always compared with a full image. The above-described movement of the heart is not corrected thereby, so the procedure is not unconditionally suitable.

Image merging of morphological imaging methods (for example CT) using functional 3D methods (MRI/Spect/PET) is known. Some of these additional measurements are laborious or very expensive. Moreover they are acquired with additional dedicated systems and are not available as up-to-date results in the intracardiac catheter laboratory.

WO 02/061444 A2 discloses a method for automatically registering a series of two-dimensional images of the heart that are successively taken. These are MR perfusion images. For improved registration, one parameter respectively is calculated between two successive images, which parameter reflects the success of the registration process. During registration the pixels, which anatomically correspond to each other, of successive images are in each case displaced to the same image coordinates in the image plane by way of a transformation.

EP 1 280 105 A2 relates to a method and to a device for registering two 3D image data records of an imaging object provided with a plurality of markers contained in the 3D image data records. This type of registration can be used for example in digital subtraction angiography.

DE 195 41 500 A1 describes a method for reconstructing single layer images from a three-dimensional volume data record which, for example, has been produced by way of spiral scanning using a computer tomograph.

### SUMMARY OF THE INVENTION

The object of the present invention is to provide a method and a device for visualizing objects, in particular non-rigid objects, in which relative displacements of the object, such as a relative displacement of organs in the case of slight respiratory movements or movements of the heart, are compensated during visualization.

This object is achieved by the method and by the device for visualizing objects with the features of the independent claims. Advantageous developments are defined in the subclaims.

Images of, for example, an angiographic series are preferably registered by a C-arm X-ray apparatus for functional evaluation or subsequent visualization against a previously taken 3D data record. The images can be projection images or 3D reconstructions (DynaCT). In the case of DynaCT, CT-like cross-sections are produced on an angiographic C-arm system. These cross-sections allow soft tissue differentiations, so, for example, structures and organs in the body and brain, and even hemorrhages in the brain, can be detected. The images can be two-dimensional as well as three-dimensional images.

Successive images from-different cardiac cycles, but at the same cardiac phases, can preferably be evaluated with each other and the change in gray scale values over time in these images can be used for functional evaluation. The known method for X-ray projection-based perfusion imaging can be used here for example.

The present invention solves the problem of functional evaluation of an angiographic series in various ways, but in each case by way of image registration. Preference should be given to non-rigid registration although rigid registration (translation and rotation only) may also be used.

The accuracy of visualization and evaluation by elimination of the movements of the object, for example of an organ, is advantageously improved. In particular visualization in the case of back projection of results onto the surface of the registered 3D volume is improved.

### BRIEF DESCRIPTION OF THE DRAWINGS

Preferred exemplary embodiments of the invention will now be described with reference to the accompanying drawings, in which:

FIG. 1 shows a series of images with N two-dimensional images of a heart, which images are slightly displaced relative to each other as a result of respiratory movements or movements of the heart and according to a first exemplary embodiment are individually registered with respect to the respective preceding image,

FIG. 2 shows a series of images with N two-dimensional images which are slightly displaced relative to each other as a result of respiratory movements or movements of the heart and according to a second exemplary embodiment are individually registered with respect to perspective projections of a three-dimensional volume,

FIG. 3 shows a schematic diagram of a device for visualizing objects according to the present invention, and

FIG. 4 shows a view of superimposition of the N two-dimensional images of the heart which are slightly displaced relative to each other as a result of respiratory movements or movements of the heart and according to the prior art are not individually registered.

### DETAILED DESCRIPTION OF THE INVENTION

A first exemplary embodiment of the present invention will be described hereinafter with reference to the drawings.

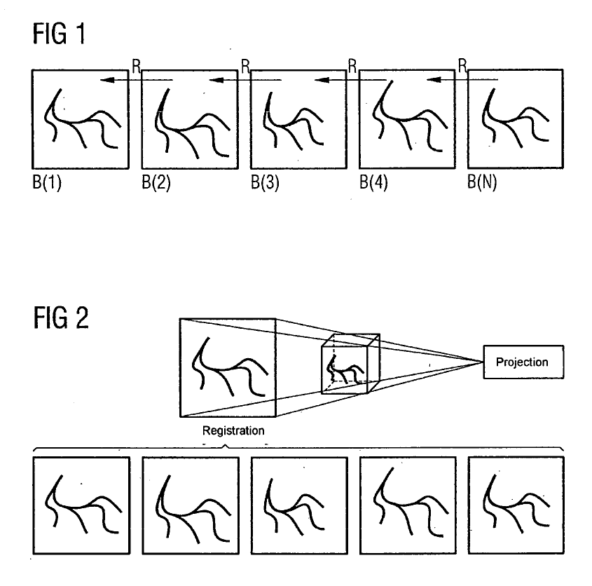

FIG. 1 shows a series of images with N two-dimensional images (B(1), B(2), . . . , B(N) of a heart, which images are slightly displaced relative to each other as a result of respiratory movements or movements of the heart and according to an exemplary embodiment that is not being claimed, are individually registered with respect to the respective preceding image.

With the method first of all the series of N two-dimensional images (B(1), B(2), . . . , B(N) is created, the images being taken successively. The first image is B(1) and the last image B(N). The two-dimensional images B(1), B(2), . . . , B(N) are each conceived as two-dimensional image data records according to the present invention. In general image information is interpreted in digital or analog form as image data records which can be stored or visualized on a volatile or non-volatile data storage medium. In particular X-ray methods, in which two-dimensional X-ray images of the object are taken by means of fluoroscopic transillumination, are suitable as methods for creating the series of images. In this case a contrast medium for example is injected into the blood vessels which are then easily visible in the two-dimensional X-ray images. A C-arm X-ray apparatus is preferably used for taking the two-dimensional X-ray images.

Each image B(n) of the image series, where n=1, 2, . . . , N, is then individually registered by way of a respective transformation matrix R. In the case of the first exemplary embodiment each image B(n) of the series of images is individually registered with respect to the immediately preceding image B(n-1) of the series of images in each case.

In this connection the individual two-dimensional images of the contrast medium series are each registered with respect to each other, so ultimately a fixed point in any desired image always corresponds to the same point in another image. The transformation matrix R[B(n)] is expediently determined for n=2, 3, . . . , N for this purpose in order to register all images with respect to each other. Each image is preferably registered simply for the transformation of its predecessor.

The contrast medium-filled vessels for example, which stand out very well against the image background, are suitable as points of reference for registration.

This procedure can take place for the entire series or a selection of images, in particular for images on which the vessels may be seen. Artifacts, especially in the known method for X-ray projection-based perfusion imaging, owing to vessel movement may be eliminated thereby. One possibility here is to limit oneself to full images of the vessels. The visible catheter may also be used in the blush phase, i.e. when it is no longer possible to see any vessels.

An exemplary embodiment of the present invention will be described hereinafter with reference to the drawings.

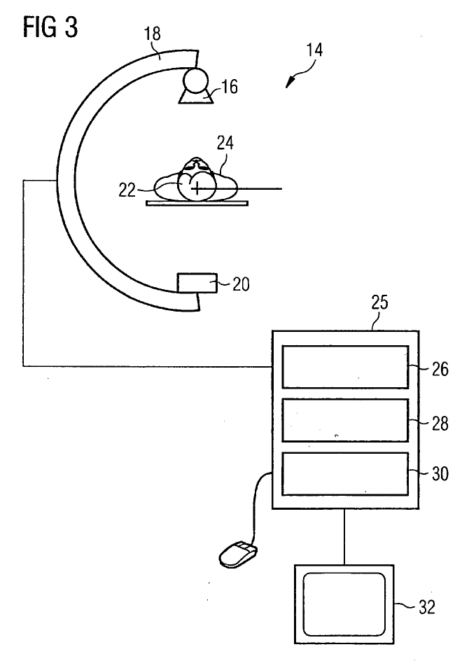

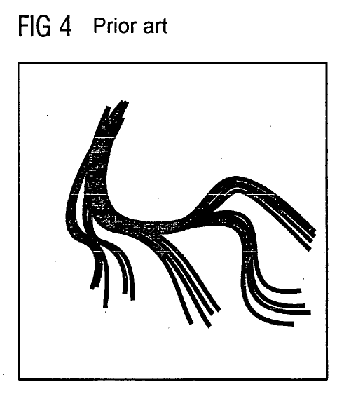

FIG. 2 shows a series of images with N two-dimensional images which have been slightly displaced relative to each other by respiratory movements or movements of the heart and according to the second exemplary embodiment are individually registered with respect to perspective projections of a three-dimensional volume.

Firstly, before automatic determination of the functional parameters, a three-dimensional anatomical data record V of the heart is created. Of course a pre-operatively taken CT-data record may also be used. In particular a fluoroscopic X-ray method, a computed tomography method (CT) such as a cardiac CT or cardiac DynaCT, a three-dimensional angiography method, a three-dimensional ultrasound method, a positron emission tomograph method (PET) or a magnetic resonance tomography method (MRT) are suitable as methods.

Similar to as in the first exemplary embodiment, a series of N two-dimensional images (B(1), B(2), . . . , B(N) is also created, which images are successively taken. The two-dimensional series of images for example contains angiographic data for functional evaluation. This method can take place in monoplan or biplan mode.

In the following step the individual images of the image series are individually registered with respect to the three-dimensional data record V of the object. If the series has N images B(1) to B(N) and Rv[B(n)] designates a transformation matrix of the image B(n) to be registered with respect to V, Rv[B(n)] is expediently determined for n=1, 2, . . . , N. Since all images B(n) are accordingly registered with respect to V, ultimately all images are,also registered with respect to each other again.

If the parameters of projection of the three-dimensional image data record for registration are not known via the unit parameters, the contrast medium-filled vessels for example, which stand out very well against the image background, are again suitable here as points of reference for registration.

The angiographically obtained functional parameters from the two-dimensional images B(1), B(2), . . . , B(N) are evaluated in the final step.

A combination of the angiographically obtained two-dimensional, functional parameters (for example X-ray projection-based perfusion) together with a three-dimensional data record is described here which shows the morphology of the examined organ (for example by DynaCT or CT).

Compared with the first exemplary embodiment the second exemplary embodiment also provides an advantage in visualization, in that the images (B(1), B(2), . . . , B(N) of the series of images may be displayed superimposed with projections which have been extracted from the three-dimensional data record V of the object. The functional values accordingly determined by way of the evaluation method can therefore be projected back onto the surface of the registered volume.

Two-dimensional projections of the object are thus extracted from the three-dimensional data record V, as is indicated in FIG. 2. The parameters of projection are known via the unit parameters, for example if the three-dimensional data record V is created on the same unit with which the two-dimensional images (B(1), B(2), . . . , B(N) that are to be registered are taken. Thus for example a volume can be generated on an angio unit using cardiac DynaCT.

Once the morphological 3D data record and evaluation of the functional, angiographically taken images exist and have been registered with each other, they can be visualized in the form of an image fusion. Known methods of 2D-3D registration are used here.

Several types of visualization are possible:
3D data record, transparent, 2D-functional data record, non-transparent,
2D-functional data record, transparent, 3D data record non-transparent,
stretching of the 2D-functional data record to the 3D data record,
2D-functional data record is either the static or the dynamic version.

In the case of a dynamic magnetic resonance tomography data record (MRT), said data record may be registered with the dynamic, functional images and visualized.

In the case of bi-plan recording of angiographic images, the functional images can be registered with the 3D data record in both planes and visualized therewith.

Functional evaluation can be based on simple parameters, such as the time of washing in/out the contrast medium in the vessel or myocardium, referred to as the mean transit time, as well as more complex parameters, such as perfusion values, blood flow values in the coronary arteries and other variables, and also derived variables, such as degrees of perfusion.

The method described here is also applicable to all other organs, especially the brain, or, in the case of other diseases (tumors, AVM=arteriovenous malformation) in the body. It is not restricted to just the heart. ECG triggering or respiratory triggering may be omitted in the case of non-moving organs.

FIG. 3 shows a schematic view of a device for visualizing objects according to the present invention. The device has an apparatus 14 for creating a series of image data records, which images are successively taken, and an apparatus 25 which individually registers each individual image data record of the series.

The apparatus 14 in this exemplary embodiment is an X-ray unit 14 with a connected appliance with which the fluoroscopic X-ray images are created. The X-ray apparatus 14 is a C-arm apparatus with a C-arm 18, on which C-arm 18 an X-ray tube 16 and an X-ray detector 20 are provided. The apparatus can, for example, be the Axiom Artis dFC belonging to Siemens AG, Medical Solutions, Erlangen, Germany. The patient 24 is lying in the field of view of the X-ray unit. Reference numeral 22 designates an object inside the patient 24 which is the intended target of the intervention, for example the liver, heart or brain. Connected to the X-ray unit is a computer 25 which in the illustrated example controls the X-ray unit and undertakes image processing and image registration. These two functions can however also be implemented separately. In the illustrated example the C-arc movement, and taking of intra-operative X-ray images, is controlled by a control module 26.

A pre-operatively taken three-dimensional image data record V can be stored in a storage device 28, it being possible to use the data record in the inventive method according to the second exemplary embodiment and the modification thereof described above.

The series of image data records comprising the two-dimensional X-ray images or the three-dimensional image data records can be registered in an arithmetic module 30 according to the method of the second exemplary embodiment and the modification thereof described above.

In the arithmetic module 30, the two-dimensional X-ray images according to the second exemplary embodiment can be superimposed with the projections from the three-dimensional image data record and the thus merged image is displayed on a screen 32.

The arithmetic module 30 is also capable of creating 3D reconstructions by means of DynaCT.

The present invention is not restricted to the illustrated embodiments; instead modifications are also incorporated by the scope of the invention which is defined by the accompanying claims.

1.-6. (canceled)

7. A method for visualizing an object of a patient under a medical examination, comprising:
generating a three-dimensional image data record of the object;
successively recording a series of two-dimensional image data records of the object;
individually registering each of the successively recorded two-dimensional image data records with the three-dimensional image data record;
evaluating a parameter of the successively recorded two-dimensional image data records;
extracting a further series of two-dimensional image data records from the three-dimensional image data record based on the evaluation;
superimposing the extracted two-dimensional image data records with the successively recorded two-dimensional image data records; and
visualizing the object with the superimposed image data records for the medical examination.

8. The method as claimed in claim 7, wherein the object is a soft tissue or a soft organ of the patient.

9. The method as claimed in claim 7, wherein the successively recorded two-dimensional image data records record displacements of the object with respective to each other as a result of a respiration or a heart movement of the patient.

10. The method as claimed in claim 9, wherein the displacements are compensated in the superimposed image data records.

11. The method as claimed in claim 7, wherein the parameter is selected from the group consisting of: a mean transit time of a contrast medium in a vessel or a myocardium of the patient, a perfusion value, and a degree of perfusion.

12. The method as claimed in claim 7, wherein the extracted two-dimensional image data records are transparently superimposed on the successively recorded two-dimensional image records that are non-transparent.

13. The method as claimed in claim 7, wherein the three-dimensional image data record is generated from a method selected from the group consisting of: CT, MR, and ultrasound.

14. A device for visualizing an object of a patient under a medical examination, comprising:
an image recording device that successively records a series of two-dimensional image data records of the object;
a storage device that stores a three-dimensional image data record of the object that is pre-operatively generated; and
a computer that:
individually registers each of the successively recorded two-dimensional image data records with the three-dimensional image data record,
evaluates a parameter of the successively recorded two-dimensional image data records,
extracts a further series of two-dimensional image data records from the three-dimensional image data record based on the evaluation, and
superimposes the extracted two-dimensional image data records with the successively recorded two-dimensional image data records for visualizing the object.

15. The device as claimed in claim 14, wherein the object is a soft tissue or a soft organ of the patient.

16. The device as claimed in claim 14, wherein the successively recorded two-dimensional image data records record displacements of the object with respective to each other as a result of a respiration or a heart movement of the patient.

17. The device as claimed in claim 16, wherein the displacements are compensated in the superimposed image data records.

18. The device as claimed in claim 14, wherein the parameter is selected from the group consisting of: a mean transit time of a contrast medium in a vessel or a myocardium of the patient, a perfusion value,.and a degree of perfusion.

19. The device as claimed in claim 14, wherein the image recording device is a C-arm X-ray device.
